(19) United States
(12) Patent Application Publication
(10) Pub. No.: US 2026/0060833 A1
(43) Pub. Date: Mar. 5, 2026

(54) Multi-Functional Body Wrap and Sling Support Device

(71) Applicant: Deborah Child, Spokane, WA (US)

(72) Inventor: Deborah Child, Spokane, WA (US)

(21) Appl. No.: 19/038,746

(22) Filed: Jan. 28, 2025

Related U.S. Application Data
(60) Provisional application No. 63/688,441, filed on Aug. 29, 2024.

(51) Int. Cl. A61F 5/37; A61G 7/10

(52) U.S. Cl. CPC A61F 5/3715; A61G 7/1023

(57) ABSTRACT

A multifunctional body part wrap device designed to assist individuals with mobility, therapeutic needs, or patient transfers is disclosed. The device includes a wrap body made of durable, tear-resistant material, configurable in various sizes and shapes to accommodate different parts or limbs of the body. A closure mechanism such as hook-and-loop fasteners is used for secure wrapping of the device around a body part (i.e., limb) and at least one handle is attached to the body via sewn seams for providing movement and positioning by the user or a caregiver. One embodiment includes a plurality of apertures and a plurality of adjustable straps to synchronize movement of multiple limb (i.e., arm or leg) wraps for bilateral use. The device can also function as a padded sling support with sewn covers to prevent twisting and reduce friction for patient comfort during transfers.

### CROSS-REFERENCE TO RELATED APPLICATION

The present application claims priority to, and the benefit of, U.S. Provisional Application No. 63/688,441 which was filed on Aug. 29, 2024, and is incorporated herein by reference in its entirety.

### FIELD OF THE INVENTION

The present invention generally relates to ergonomic limb (i.e., arm or leg) wrap devices. More specifically, the invention relates to a limb wrap designed to assist in positioning, mobility, and therapeutic applications for individuals with disabilities, injuries, or limited mobility. The limb wrap comprises a body made of durable, soft, and tear-resistant material, configured to wrap around the leg, arm, or other body parts. The device features a closure mechanism, such as a hook-and-loop fastener or snap buttons, for secure and adjustable fastening. At least one repositionable handle is attached (i.e., sewn) and enables users or caregivers to lift, move, or position (i.e., maneuver) the leg with minimal effort. The device can also be adapted for use on arms, any other body part, or as a padded sling support for patient transfers. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals who suffer from physical disabilities, temporary injuries, or chronic conditions often face significant challenges in maintaining proper leg alignment and mobility. For individuals who face leg injury or surgery, may experience difficulty in adjusting their legs into a comfortable position. Misalignment of the hips and legs can lead to severe pain, impaired posture, and secondary complications such as muscle atrophy, ligament deterioration, and joint stiffness. Such issues are particularly common among individuals with limited mobility, including the elderly, patients recovering from surgery, and those with neurological or musculoskeletal disorders.

Individuals often need help from caregivers for movement of their legs and caregivers also often struggle to assist the individuals in moving or repositioning their legs due to the lack of ergonomic, supportive tools. Existing devices, such as slings or supports, often fail to provide adequate comfort, stability, or adjustability. This can result in increased physical strain for caregivers and heightened risks of injury for patients, including skin irritation, pressure sores, and tissue damage caused by friction or improper device use. Individuals desire a limb (i.e., arm or leg) wrap and sling support device that provides physical support and comfort while also providing adaptability for different users, contexts, and body sizes.

Therefore, there exists a long-felt need in the art for a limb wrap device that provides an ergonomic solution for individuals with mobility challenges. There is a long-felt need for a device that minimizes discomfort and prevents complications such as muscle atrophy, ligament deterioration, and hip misalignment caused by improper leg positioning. Additionally, there is a long-felt need for a versatile device that can be adapted for different body parts and used in a variety of environments, including therapeutic settings, daily activities, and wet conditions such as showers. Furthermore, there is a long-felt need for a limb wrap device that is user-friendly, easy to adjust, and equipped with handles to assist caregivers in safely lifting, moving, or repositioning the user's limb. More specifically, there exists a long-felt need in the art for an improved limb (i.e., arm or leg) wrap device that enables the user to stabilize their legs and adjust the hips and legs into the desired position. Finally, there is a long-felt need for a device that is durable, customizable, and makes it easy and convenient for a user to move their leg, arm, or any other body part into the desired position.

The subject matter disclosed and claimed herein, in one embodiment, comprises a limb wrap device designed to assist in positioning, mobility, and therapeutic applications. The device includes a body made of a durable, soft, and tear-resistant material, available in various sizes and shapes to accommodate different parts of the body. The device features a closure mechanism, such as a hook-and-loop fastener, magnetic closure, or snap buttons, which enables the limb wrap to be securely fastened around the leg, arm, or other body parts. Additionally, the device is equipped with repositionable handles attached (i.e., sewn) which enable users or caregivers to easily lift, move, or position (i.e., maneuver) the limb into a desired location. One embodiment of the device includes apertures for accommodating adjustable straps, which can connect multiple wraps for synchronized leg movement or enhanced stability.

In this manner, the limb wrap device of the present invention accomplishes all of the foregoing objectives and provides a practical solution for individuals with limited mobility or therapeutic needs. The durable materials and ergonomic design prevent friction, pressure sores, and skin irritation during use and the repositionable handles and straps provide ease of use for caregivers and patients to position the leg in the desired position. The device can be adapted to wrap other body parts, such as arms, or function as a padded sling support for patient transfers.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a limb (i.e., arm or leg) wrap device for assisting in positioning, movement, or therapeutic use of a user's leg. The device comprises a body made of a durable, soft, and tear-resistant material, the body is configured to wrap around the leg, arm, or other body parts of the user, a hook fastener is disposed along one longitudinal end of the body and a corresponding loop fastener is disposed along the opposite or opposing longitudinal end of the body, wherein the hook and loop fasteners are configured to removably secure the limb wrap device around the leg, arm, or other body part, at least one repositionable handle is attached (i.e., sewn) to the body, the handle configured to enable the user or a caregiver to lift, move, or position (i.e., maneuver) the limb into a desired location with minimal effort.

In another aspect, a limb wrap device for therapeutic or mobility purposes is disclosed. The limb wrap device includes a body made of a material selected from, neoprene, linen, micro-suede, elastic, denim, or other washable, durable, waterproof, or tear-resistant materials, wherein the body is adaptable to wrap around different parts of the body, including legs and arms, a plurality of apertures are disposed in the body, the apertures are configured to accommodate adjustable straps. The adjustable straps are removably fastened through the apertures, wherein the adjustable straps are configured to connect multiple limb wrap devices for synchronized movement of a user's legs when the limb wrap devices are worn on both limbs, and a closure mechanism for securing the limb wrap device around the leg, arm, or other body part of the user.

In another embodiment, a sling support device for patient mobility and comfort is disclosed. The device comprises a padded body, the padded body is configured to provide stability, reduce friction, and minimize skin irritation during patient transfers or movements, the padded body is further configured to be used with slings for lifting or transferring patients and to prevent friction and pressure on the skin, a plurality of straps enclosed by corresponding covers, each cover is sewn along the edges of the straps to minimize twisting or folding of the straps within the cover.

In one embodiment, a multi-functional body wrap device is disclosed. The device includes a body of varying sizes and shapes, the body made of a durable, soft, and non-stretchable material, wherein the body is adaptable to different user requirements, including use for children and adults, a fastening mechanism comprising at least one of hook and loop, snap buttons, magnetic fasteners, or other conventional fasteners, the fastening mechanism is configured to secure the wrap around a body part, a repositionable handle is configured to be attached to the body using sewn seams, wherein the handle is adjustable for optimal positioning based on the user's or caregiver's needs, and the device configured for use in therapeutic settings, daily mobility, or as a sling support, with options for customization in material, thickness, and size to suit individual users.

In another aspect, the repositionable handle is adjustable to different positions along the body via the sewn seams, enabling customization for varying user needs or therapeutic purposes.

In yet another aspect, the adjustable straps are made of non-stretchable material and include quick-release buckles, enabling easy detachment and reattachment during use for improved caregiver convenience.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a limb (i.e., arm or leg) wrap device of the present invention in accordance with the disclosed architecture;

FIG. 2 illustrates a perspective view of another embodiment of the limb wrap device of the present invention in accordance with the disclosed structure;

FIG. 3 illustrates a perspective view showing a user wearing the limb wrap device of FIG. 2 in accordance with one embodiment of the present invention; and

FIG. 4 illustrates another embodiment of the limb wrap device of the present invention to be used as a sling support in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a limb (i.e., arm or leg) wrap device that provides an ergonomic solution for individuals with mobility challenges. There is a long-felt need for a device that minimizes discomfort and prevents complications such as muscle atrophy, ligament deterioration, and hip misalignment caused by improper leg positioning. Additionally, there is a long-felt need for a versatile device that can be adapted for different body parts and used in a variety of environments, including therapeutic settings, daily activities, and wet conditions like showers. Furthermore, there is a long-felt need for a limb wrap device that is user-friendly, easy to adjust, and equipped with handles to assist caregivers in safely lifting, moving, or repositioning the user. More specifically, there exists a long-felt need in the art for an improved limb wrap device that enables the user to stabilize their legs and adjust the hips and legs into the desired position. Finally, there is a long-felt need for a device that is durable, customizable, and makes it easy and convenient for a user to move the leg, arm, or any other body part into the desired position.

The present invention, in one exemplary embodiment, is a multi-functional body wrap device. The device includes a body of varying sizes and shapes, the body made of a durable, soft, and non-stretchable material, wherein the body is adaptable to different user requirements, including use for children and adults, a fastening mechanism comprising at least one of hook and loop, snap buttons, magnetic fasteners, or other conventional fasteners, the fastening mechanism is configured to secure the wrap around a body part, a repositionable handle is configured to be attached to the body using, for example, sewn seams, wherein the handle is adjustable for optimal positioning based on the user's or caregiver's needs.

Reference will now be made in detail to the present preferred embodiments of the invention, examples of which are illustrated in the accompanying drawings. Wherever possible, the same reference numerals are used in the drawings and the description to refer to the same or like parts.

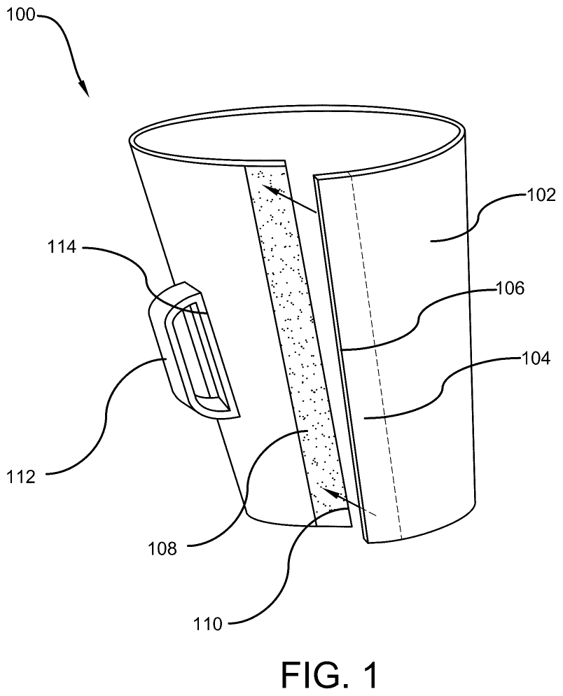

Referring initially to the drawings, FIG. 1 illustrates a perspective view of one potential embodiment of a limb (i.e., arm or leg) wrap device of the present invention in accordance with the disclosed architecture. The limb wrap device 100 of the present invention is designed as an ergonomic limb wrap for individuals who need assistance in positioning their legs (i.e., limbs), whether for mobility, therapeutic, or other functional purposes. More specifically, the limb wrap device 100 includes a body 102 made of durable, soft, and tear-resistant material. The body 102 can be of different sizes and shapes, and/or include stretchable (i.e., elastic) material, to fit around different parts of the body and more specifically, different parts of the limb.

The body 102 includes a hook fastener portion 104 along one longitudinal end 106 thereof and a corresponding loop fastener portion 108 along the opposite longitudinal end 110 thereof. The hook fastener portion 104 and the loop fastener portion 108 are configured to removably fasten to each other to wrap the limb wrap or corset device 100 around leg, arm, or any other body part of a user. It should be noted that any other fastening means such as snap buttons, magnetic fasteners, or any other conventional fasteners can be used for wrapping and securing the limb wrap or corset device 100.

At least one handle or arm loop 112 is disposed on the exterior of the body 102 for enabling the wearer of the device 100 or a caregiver to easily lift, move, or position (i.e., maneuver) the limb into a desired location with minimal effort. It is to be appreciated that the handle 112 is sized for the wearer (i.e., sans hands) of the device 100 to place their arm (i.e., forearm) through the handle 112. The handle 112 is repositionable and is secured using a plurality of sewn seams 114. The device 100 can be adapted to create wraps for arms or other body parts to meet the requirements of different users. The device 100 has therapeutic use for individuals recovering from injury, surgery, or those with disabilities requiring assistance in leg or arm positioning.

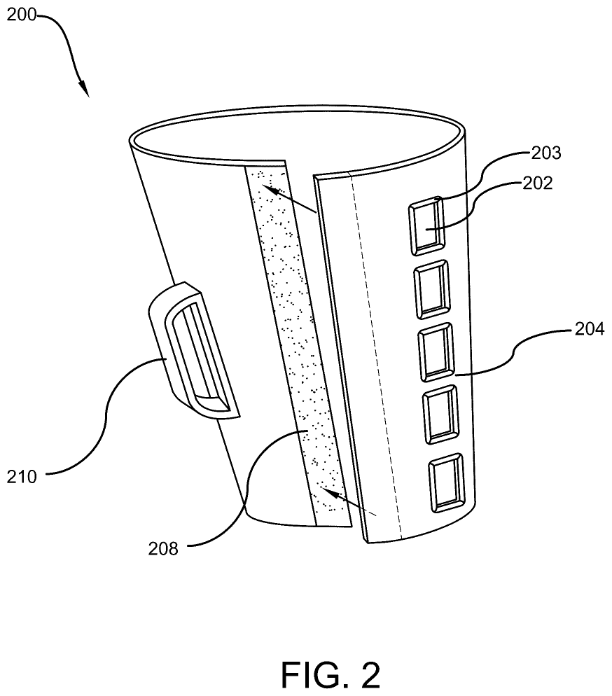

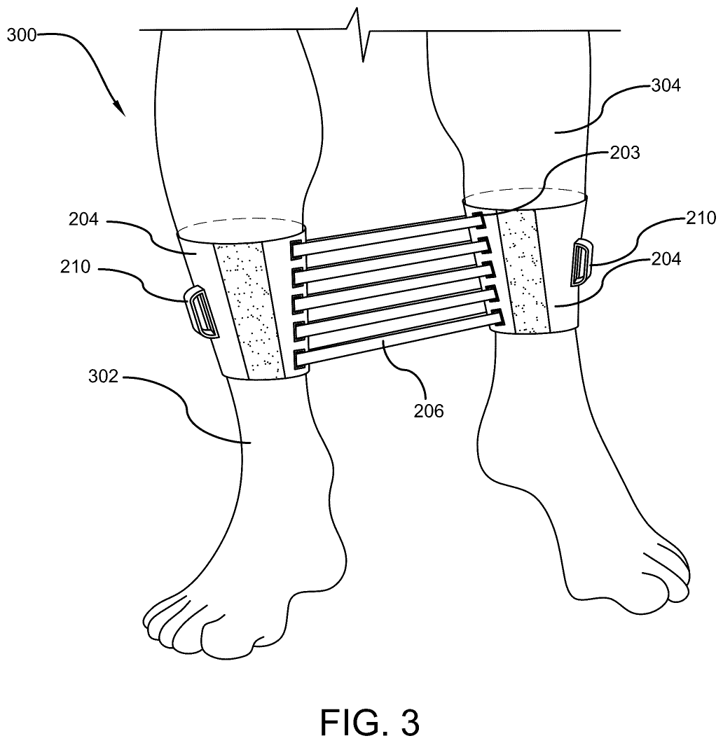

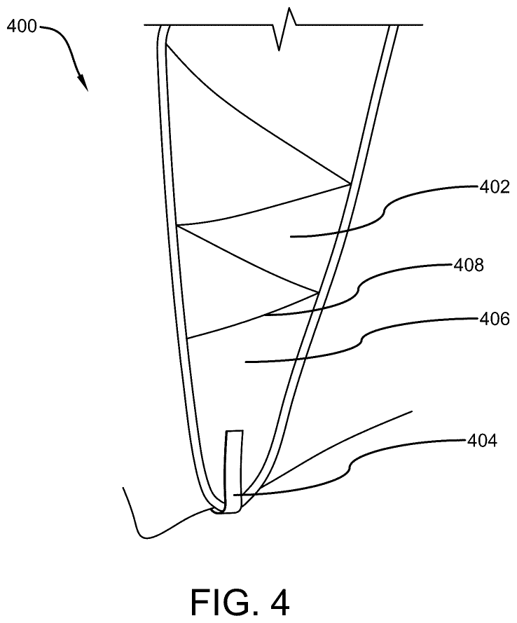

FIG. 2 illustrates a perspective view of another embodiment of the limb wrap device of the present invention in accordance with the disclosed structure. In the present embodiment, the ergonomic limb wrap and sling support device 200 includes a plurality of apertures 202 disposed in the body 204 of the device 200. The apertures 202 and grommets 203 therearound are used for accommodating and securing adjustable straps 206 wherein the adjustable straps 206 as shown in FIG. 3 are used for removably fastening two limb wrap devices 200 together, such as when the limb wrap devices are worn on two legs by a user. It is to be appreciated that the adjustable straps 206 can be attached to a secured or fixed object for fixing the device 200 and the contained limb in a stationary orientation. Alternatively, the adjustable straps 206 can be attached to a sling (FIG. 4). The sling can be secured around a neck of the user of the device 200 for holding, for example, the arm in a suspended orientation.

The limb wrap device 200 includes a closure mechanism 208 such as hook and loop for securing the limb wrap device 200 around leg, arm, or any other body part of the user. The repositionable handle 210 is used for holding to easily lift, move, or position (i.e., maneuver) the limb into a desired location with minimal effort. The combination of adjustable straps 206 and the handle 210 makes easy for both users and caregivers to manipulate the wrap for movement of the user using the device 200.

FIG. 3 illustrates a perspective view showing a user wearing the limb wrap device 200 of FIG. 2 in accordance with one embodiment of the present invention. As illustrated, a user 300 wears the limb wrap device 200 around each leg 302, 304 and each multi-functional body wrap device 200 is secured using the closure mechanism 208 and the adjustable straps 206 help in synchronization of movement of both the legs 302, 304. The ergonomic limb wrap and sling support device 100 can be easily removed by unfastening the closure mechanism 208 and the adjustable straps 206 can also be detached from the corresponding apertures 202 and grommets 203. Each strap can include a quick-release buckle for enabling easy detachment and reattachment of the straps during use for wrapping the device around a body part of a user.

FIG. 4 illustrates another embodiment of the limb wrap device of the present invention to be used as a sling support in accordance with the disclosed structure. In the present embodiment, the device 400 includes the padded body 402 which covers a plurality of straps 404 such as used in patient slings and each strap 404 is enclosed by a corresponding cover 406. The body 402 is strategically sewn along edges 408 to minimize twisting or folding of each strap 404 inside the sleeve or cover 406, thereby providing stability and comfort. The thickness of padded body 402 can range from 2 to 4 inches, depending on patient needs and comfort levels. Thicker pads may be used for larger patients whose legs have atrophied but require more support for their backsides. The device can be stored in a clean and dry location to prevent mold or material degradation. The limb wrap device reduces friction and pressure on the skin and prevents skin tearing or irritation caused by the edges of slings or straps during use.

In some embodiments of the present invention, the multi-functional body wrap device is wrapped around the leg, arm, or any other body area needing the support. The wrap device is snugly fastened using the hook and loop or other closure mechanism. Then, the position of the handles is confirmed in the correct position for ease of use, based on the desired movement or assistance required. For the sling support, the padded body cover is used for inserting the sling strap therein and the padded body is placed under a user's body for enabling the sling straps to be used for moving or transferring a patient.

The multi-functional body wrap device of different embodiments can be made of one or more of neoprene, linen, micro-suede, elastic, denim, or any other washable, durable, waterproof, stain-resistant, and tear-resistant materials.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “limb (i.e., arm or leg) wrap device”, “leg corset device”, “multi-functional body wrap device”, “ergonomic limb wrap and sling support device”, “ergonomic multi-functional body wrap and sling support device”, and “device” are interchangeable and refer to the multi-functional limb wrap and sling support device 100, 200, 300, 400 of the present invention.

Notwithstanding the forgoing, the multi-functional body wrap and sling support device 100, 200, 300, 400 of the present invention can be of any suitable size and configuration as is known in the art without affecting the overall concept of the invention, provided that it accomplishes the above stated objectives. One of ordinary skill in the art will appreciate that the multi-functional body wrap and sling support device 100, 200, 300, 400 as shown in the FIGS. are for illustrative purposes only, and that many other sizes and shapes of the multi-functional body wrap and sling support device 100, 200, 300, 400 are well within the scope of the present disclosure. Although the dimensions of the multi-functional body wrap and sling support device 100, 200, 300, 400 are important design parameters for user convenience, the multi-functional body wrap and sling support device 100, 200, 300, 400 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A limb wrap device comprising:
a limb wrap;
a body;
a fastener; and
a handle;
wherein said body having an elastic material;
wherein said fastener is selected from the group consisting of a snap button, a magnetic fastener, and a hook and loop fastener to removably fasten to each other for selectively fastening said limb wrap around a user's limb; and
further wherein said handle disposed on an exterior of said body for grasping and maneuvering of said limb wrap.

2. The limb wrap device of claim 1, wherein said handle is repositionable.

3. The limb wrap device of claim 2, wherein said handle having a plurality of sewn seams for securing to said body.

4. The limb wrap device of claim 3, wherein said hook and loop fastener having a hook fastener portion along one longitudinal end of said body and a loop fastener portion along an opposing longitudinal end of said body.

5. The limb wrap device of claim 3, wherein said body having a plurality of apertures and a plurality of straps, and further wherein said plurality of straps securable to another plurality of apertures of another body of another limb wrap for securing said limb wrap to said another limb wrap.

6. The limb wrap device of claim 3, wherein said body having a plurality of apertures and a plurality of straps, and further wherein said plurality of straps securable to a fixed object for fixing said limb wrap in a stationary orientation.

7. The limb wrap device of claim 3, wherein said body having a plurality of apertures and a plurality of straps, and further wherein said plurality of straps securable to a sling for suspending a limb in said limb wrap.

8. A plurality of limb wrap devices comprising:
a first limb wrap having a first body, a first fastener, and a first handle;
a second limb wrap having a second body, a second fastener, and a second handle;
wherein said first fastener and said second fastener are selected from the group consisting of a snap button, a magnetic fastener, and a hook and loop fastener to removably fasten to each other for selectively fastening said first limb wrap around a user's first limb and selectively fastening said second limb wrap around a user's second limb; and
wherein said first handle disposed on an exterior of said first body for grasping and maneuvering of said first limb wrap;
wherein said second handle disposed on an exterior of said second body for grasping and maneuvering of said second limb wrap;
wherein said first body having a first plurality of apertures;
wherein said second body having a second plurality of apertures; and
further wherein at least one of said first body and said second body having a plurality of straps for securing said first limb wrap to said second limb wrap.

9. The plurality of limb wrap devices of claim 8, wherein said first body and said second body having elastic material.

10. The plurality of limb wrap devices of claim 8, wherein said first handle and said second handle are arm loops.

11. The plurality of limb wrap devices of claim 8, wherein said first handle having a first plurality of sewn seams for securing to said first body.

12. The plurality of limb wrap devices of claim 11, wherein said second handle having a second plurality of sewn seams for securing to said second body.

13. The plurality of limb wrap devices of claim 8, wherein said hook and loop fastener of said first fastener having a hook fastener portion along one longitudinal end of said first body and a loop fastener portion along an opposing longitudinal end of said first body.

14. The plurality of limb wrap devices of claim 13, wherein said hook and loop fastener of said second fastener having a hook fastener portion along one longitudinal end of said second body and a loop fastener portion along an opposing longitudinal end of said second body.

15. A limb wrap device comprising:
a limb wrap;
a body;
a fastener; and
a handle;
wherein said fastener is selected from the group consisting of a snap button, a magnetic fastener, and a hook and loop fastener to removably fasten to each other for selectively fastening said limb wrap around a user's limb;
wherein said handle disposed on an exterior of said body for grasping and maneuvering of said limb wrap;
wherein said body having a plurality of apertures and a plurality of straps;
wherein said plurality of straps securable at one end to said body;
wherein said plurality of straps securable at another end to an object; and
further wherein said object is selected from the group consisting of another body of another limb wrap, a fixed object, and a sling.

16. The limb wrap device of claim 15, wherein said handle is an arm loop.

17. The limb wrap device of claim 16, wherein said handle having a plurality of sewn seams for securing to said body.

18. The limb wrap device of claim 17, wherein said hook and loop fastener having a hook fastener portion along one longitudinal end of said body and a loop fastener portion along an opposing longitudinal end of said body.

19. The limb wrap device of claim 18, wherein said body having an elastic material.

20. The limb wrap device of claim 18, wherein said body having a material selected from the group consisting of a neoprene, a linen, a micro-suede, an elastic, and a denim.
